(19) United States
(12) Patent Application Publication
(10) Pub. No.: US 2006/0142359 A1
(43) Pub. Date: Jun. 29, 2006

(54) Cyclopenta[c]isoxazole-3-amines used as protective agents for materials

(76) Inventors: Rainer Bruns, Leverkusen (DE); Gunther Eberz, Odenthal (DE); Wolfgang Kreiss, Bergisch Gladbach (DE); Oliver Kretschik, Koln (DE); Martin Kugler, Leichlingen (DE); Hermann Uhr, Leverkusen (DE); Erasmus Vogl, Leverkusen (DE); Peter Wachtler, Krefeld (DE)

Correspondence Address: Lanxess Corporation;Law & Intellectual Property Department, 111 Ride Park West Drive, Pittsburgh, PA, 15275-1112, US

(21) Appl. No.: 10/537,242

(22) PCT Filed: Nov. 22, 2003
(86) PCT No.: PCT/EP03/13140

§ 371 Date: Feb. 16, 2006

(30) Foreign Application Priority Data
Dec. 2, 2002 (DE) 102 56 186.9

(51) Int. Cl. A01N 43/80; C09D 5/16; C07D 261/20

(52) U.S. Cl. 514/379; 548/241; 523/122

(57) ABSTRACT

The novel 2-oxyamino-1-cyclopentene-1-nitriles of the formula (I) 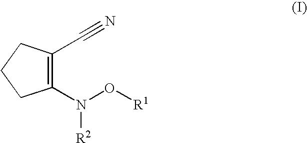
in which the substituents R¹ and R² are as defined in the description are highly suitable for protecting industrial materials against infestation and destruction by microorganisms.

The present invention relates to novel cyclopenta[c]isoxazole-3-amines, to processes for their preparation, to their use for controlling unwanted microorganisms and to novel mixtures of cyclopenta[c]isoxazole-3-amines with other active compounds.

Very few cyclopenta[c]isoxazole-3-amines are known from the literature (cf. G. Gerali et al., Farmaco Edizione Scientifica 1969, 24, 1105-1114), and an action against material-destroying organisms has not been described.

We have found novel cyclopenta[c]isoxazole-3-amines which, surprisingly, have excellent bactericidal action. Owing to their antibacterial and antifungal action, the novel cyclopenta[c]isoxazole-3-amines are, on their own or in a mixture with one another, particularly suitable for controlling microorganisms in and on industrial materials.

The present invention provides cyclopenta[c]isoxazole-3-amines of the formula (I) 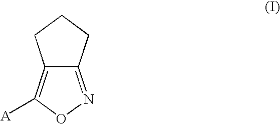
in which
A represents a radical

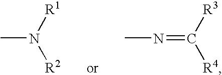

in which
R¹ and R² independently of one another represent hydrogen, halogen, cyano, nitro or represent in each case optionally substituted alkyl, alkenyl, alkynyl, aryl, heterocyclyl, —COR⁵, —CONR⁶, —CSNR⁷ or —SO₂R⁸,
   where
R⁵ to R⁸ independently of one another represent in each case optionally substituted alkyl, alkenyl, alkynyl, aryl or heterocyclyl,
   and
R³ and R⁴ independently of one another represent hydrogen, or represent in each case optionally substituted alkyl, alkenyl, alkynyl, aryl or heterocyclyl,
   and their salts and acid addition compounds.

In the definitions of the substituents R¹ to R⁸, the saturated or unsaturated hydrocarbon radicals, such as alkyl, alkenyl or alkynyl, are in each case straight-chain or branched and unsubstituted or mono- to polysubstituted by identical or different substituents, including in combination with heteroatoms, such as in alkoxy, haloalkoxy, haloalkylthio or alkylthio, and in composite terms, such as alkyl- or dialkylamino.

In the alkyl- and dialkylamino substituents mentioned, the alkyl radicals can in each case be identical or different.

Aryl represents aromatic mono- or polycyclic hydrocarbon rings which are unsubstituted or mono- to polysubstituted by identical or different substituents, such as, for example, phenyl, naphthyl, anthranyl, phenanthranyl, preferably phenyl or naphthyl, in particular phenyl.

In the terms haloalkyl, haloalkoxy and haloalkylthio, the halogen atoms can in each case be identical or different. Halogen represents generally fluorine, chlorine, bromine, in particular fluorine or chlorine.

Heterocyclyl represents saturated and unsaturated and also aromatic cyclic compounds in which at least one ring member is a heteroatom, i.e. an atom different from carbon, which compounds are unsubstituted or mono- to polysubstituted by identical or different substituents. If the ring contains a plurality of heteroatoms, these can be identical or different. Preferred heteroatoms are oxygen, nitrogen or sulfur. If appropriate, the cyclic compounds form, together with further carbocyclic or heterocyclic fused-on or bridged rings, a polycyclic ring system. A polycyclic ring system may be attached via the heterocyclic ring or a fused-on carbocyclic ring. Preference is given to mono- or bicyclic ring systems, in particular mono- or bicyclic aromatic ring systems. Preferred heterocyclyl radicals are pyridyl, pyrimidyl, thienyl, furyl and pyrryl.

Preference is given to compounds of the formula (I),

in which

R¹ and R² independently of one another represent hydrogen, halogen, cyano, nitro or in each case optionally substituted C₁-C₈-alkyl, C₂-C₈-alkenyl, C₂-C₈-alkynyl, phenyl or heterocyclyl, or represent a radical —COR⁵, CONR⁶, —CSNR⁷ or —SO₂R⁸,
   where
R⁵ to R⁸ independently of one another represent hydrogen, halogen, cyano, nitro or represent in each case optionally substituted C₁-C₈-alkyl, C₂-C₈-alkenyl, C₂-C₈-alkynyl, phenyl or heterocyclyl, and
R³ and R⁴ independently of one another represent hydrogen, halogen, cyano, nitro or represent in each case optionally substituted C₁-C₈-alkyl, C₂-C₈-alkenyl, C₂-C₈-alkynyl, phenyl or heterocyclyl.

Particularly preferred are compounds of the formula (I) in which
R¹ and R² independently of one another represent hydrogen, halogen, cyano, nitro, or represent C₁-C₈-alkyl, C₂-C₈-alkenyl, or C₂-C₈-alkynyl which are in each case optionally mono- or polysubstituted by identical or different substituents from the group consisting of halogen, nitro, cyano, phenyl, C₁-C₆-alkoxy, C₁-C₆-haloalkoxy having 1 to 9 identical or different halogen atoms, C₁-C₆-alkylthio, C₁-C₆-haloalkylthio having 1 to 9 identical or different halogen atoms, C₁-C₆-acyl, C₁-C₆-acyloxy, C₁-C₆-alkoxycarbonyl, amino, C₁-C₆-alkylamino, di-C₁-C₆-alkylamino, phenylamino and diphenylamino;
   or represent phenyl which is optionally mono- or polysubstituted by identical or different substituents from the group consisting of halogen, cyano, nitro, C₁-C₅-alkyl, C₁-C₅-haloalkyl having 1 to 6 identical or different halogen atoms, C₁-C₅-alkoxy, C₁-C₅-haloalkoxy having 1 to 6 identical or different halogen atoms, C₁-C₅-alkylthio, C₁-C₅-haloalkylthio having 1 to 6 identical or different halogen atoms, amino, C₁-C₆-alkylamino, di-C₁-C₆-alkylamino, phenylamino and diphenylamino;
   or represent heterocyclyl which is optionally mono- or polysubstituted by identical or different substituents from the group consisting of halogen, cyano, nitro, C₁-C₅-alkyl, C₁-C₅-haloalkyl having 1 to 6 identical or different halogen atoms, C₁-C₅-alkoxy, C₁-C₅-haloalkoxy having 1 to 6 identical or different halogen atoms, C₁-C₅-alkylthio, C₁-C₅-haloalkylthio having 1 to 6 identical or different halogen atoms, amino, C₁-C₆-alkylamino, di-C₁-C₅-alkylamino,
   or represent —COR⁵, —CONR⁶, —CSNR⁷, —SO₂R⁸,
      where
R⁵ to R⁸ independently of one another represent hydrogen, halogen, cyano, nitro, or represent C₁-C₈-alkyl, C₂-C₈-alkenyl, C₂-C₈-alkynyl, which are in each case optionally mono- or polysubstituted by identical or different substituents from the group consisting of halogen, nitro, cyano, phenyl, C₁-C₆-alkoxy, C₁-C₆-haloalkoxy having 1 to 9 identical or different halogen atoms, C₁-C₆-alkylthio, C₁-C₆-haloalkylthio having 1 to 9 identical or different halogen atoms, C₁-C₆-acyl, C₁-C₆-acyloxy, C₁-C₆-alkoxycarbonyl, amino, C₁-C₆-alkylamino, di-C₁-C₆-alkylamino, phenylamino and diphenylamino;
   or represent phenyl which is optionally mono- or polysubstituted by identical or different substituents from the group consisting of halogen, cyano, nitro, C₁-C₅-alkyl, C₁-C₅-haloalkyl having 1 to 6 identical or different halogen atoms, C₁-C₅-alkoxy, C₁-C₅-haloalkoxy having 1 to 6 identical or different halogen atoms, C₁-C₅-alkylthio, C₁-C₅-haloalkylthio having 1 to 6 identical or different halogen atoms, amino, C₁-C₆-alkylamino and di-C₁-C₅-alkylamino;
   or represent heterocyclyl which is optionally mono- or polysubstituted by identical or different substituents from the group consisting of halogen, cyano, nitro, C₁-C₅-alkyl, C₁-C₅-haloalkyl having 1 to 6 identical or different halogen atoms, C₁-C₅-alkoxy, C₁-C₅-haloalkoxy having 1 to 6 identical or different halogen atoms, C₁-C₅-alkylthio, C₁-C₅-haloalkylthio having 1 to 6 identical or different halogen atoms, amino, C₁-C₆-alkylamino and di-C₁-C₅-alkylamino;
      and
R³ and R⁴ independently of one another represent hydrogen, halogen, cyano, nitro, or represent C₁-C₈-alkyl, C₂-C₈-alkenyl or C₂-C₈-alkynyl, which are in each case optionally mono- or polysubstituted by identical or different substituents from the group consisting of halogen, nitro, cyano, phenyl, C₁-C₆-alkoxy, C₁-C₆-haloalkoxy having 1 to 9 identical or different halogen atoms, C₁-C₆-alkylthio, C₁-C₆-haloalkylthio having 1 to 9 identical or different halogen atoms, C₁-C₆-acyl, C₁-C₆-acyloxy, C₁-C₆-alkoxycarbonyl and amino, C₁-C₆-alkylamino, di-C₁-C₆-alkylamino, phenylamino and diphenylamino;
   or represent phenyl which is optionally mono- or polysubstituted by identical or different substituents from the group consisting of halogen, cyano, nitro, C₁-C₅-alkyl, C₁-C₅-haloalkyl having 1 to 6 identical or different halogen atoms, C₁-C₅-alkoxy, C₁-C₅-haloalkoxy having 1 to 6 identical or different halogen atoms, C₁-C₅-alkylthio, C₁-C₅-haloalkylthio having 1 to 6 identical or different halogen atoms, amino, C₁-C₆-alkylamino and di-C₁-C₅-alkylamino;
   or represent heterocyclyl which is optionally mono- or polysubstituted by identical or different substituents from the group consisting of halogen, cyano, nitro, C₁-C₅-alkyl, C₁-C₅-haloalkyl having 1 to 6 identical or different halogen atoms, C₁-C₅-alkoxy, C₁-C₅-haloalkoxy having 1 to 6 identical or different halogen atoms, C₁-C₅-alkylthio, C₁-C₅-haloalkylthio having 1 to 6 identical or different halogen atoms, amino, C₁-C₆-alkylamino and di-C₁-C₅-alkylamino.

Particular preference is given to compounds of the formula (I) in which
R¹ and R² independently of one another represent hydrogen, fluorine, chlorine, bromine, cyano, nitro or represent C₁-C₆-alkyl, C₂-C₆-alkenyl or C₂-C₆-alkynyl, which are in each case optionally mono- to tetrasubstituted by identical or different substituents from the group consisting of fluorine, chlorine, bromine, nitro, cyano, phenyl, C₁-C₄-alkoxy, C₁-C₄-haloalkoxy having 1 to 7 identical or different fluorine, chlorine or bromine atoms, C₁-C₄-alkylthio, C₁-C₄-haloalkylthio having 1 to 7 identical or different fluorine, chlorine or bromine atoms, C₁-C₄-acyl, C₁-C₄-acyloxy, C₁-C₄-alkoxy-carbonyl, amino, C₁-C₄-alkylamino, di-C₁-C₄-alkylamino, phenylamino and diphenylamino;
   or represent phenyl which is optionally mono- to tetrasubstituted by identical or different substituents from the group consisting of fluorine, chlorine, bromine, cyano, nitro, C₁-C₄-alkyl, C₁-C₄-haloalkyl having 1 to 4 identical or different fluorine, chlorine or bromine atoms, C₁-C₄-alkoxy, C₁-C₄-haloalkoxy having 1 to 4 identical or different fluorine, chlorine or bromine atoms, C₁-C₄-alkylthio, C₁-C₄-haloalkylthio having 1 to 4 identical or different fluorine, chlorine or bromine atoms, amino, C₁-C₄-alkylamino and di-C₁-C₄-alkylamino;
   or represent heterocyclyl which is optionally mono- to tetrasubstituted by identical or different substituents from the group consisting of fluorine, chlorine, bromine, cyano, nitro, C₁-C₄-alkyl, C₁-C₄-haloalkyl having 1 to 4 identical or different fluorine, chlorine or bromine atoms, C₁-C₄-alkoxy, C₁-C₄-haloalkoxy having 1 to 4 identical or different fluorine, chlorine or bromine atoms, C₁-C₄-alkylthio, C₁-C₄-haloalkylthio having 1 to 4 identical or different fluorine, chlorine or bromine atoms, amino, C₁-C₄-alkylamino and di-C₁-C₄-alkylamino,
   or represent —COR⁵, —CONR⁶, —CSNR⁷ or —SO₂R⁸,
      where
R⁵ to R⁸ independently of one another represent hydrogen, fluorine, chlorine, bromine, cyano, nitro or represent C₁-C₆-alkyl, C₂-C₆-alkenyl or C₂-C₆-alkynyl which are in each case optionally mono- to tetrasubstituted by identical or different substituents from the group consisting of fluorine, chlorine, bromine, nitro, cyano, phenyl, C₁-C₄-alkoxy, C₁-C₄-haloalkoxy having 1 to 7 identical or different fluorine, chlorine or bromine atoms, C₁-C₄-alkylthio, C₁-C₄-haloalkylthio having 1 to 7 identical or different fluorine, chlorine or bromine atoms, C₁-C₄-acyl, C₁-C₄-acyloxy, C₁-C₄-alkoxy-carbonyl, amino, C₁-C₄-alkylamino, di-C₁-C₄-alkylamino, phenylamino and diphenylamino;
   or represent phenyl which is optionally mono- to tetrasubstituted by identical or different substituents from the group consisting of fluorine, chlorine, bromine, cyano, nitro, C₁-C₄-alkyl, C₁-C₄-haloalkyl having 1 to 4 identical or different fluorine, chlorine or bromine atoms, C₁-C₄-alkoxy, C₁-C₄-haloalkoxy having 1 to 4 identical or different fluorine, chlorine or bromine atoms, C₁-C₄-alkylthio, C₁-C₄-haloalkylthio having 1 to 4 identical or different fluorine, chlorine or bromine atoms, amino, C₁-C₄-alkylamino and di-C₁-C₄-alkylamino;
   or represent heterocyclyl which is optionally mono- to tetrasubstituted by identical or different substituents from the group consisting of fluorine, chlorine, bromine, cyano, nitro, C₁-C₄-alkyl, C₁-C₄-haloalkyl having 1 to 4 identical or different fluorine, chlorine or bromine atoms, C₁-C₄-alkoxy, C₁-C₄-haloalkoxy having 1 to 4 identical or different fluorine, chlorine or bromine atoms, C₁-C₄-alkylthio, C₁-C₄-haloalkylthio having 1 to 4 identical or different fluorine, chlorine or bromine atoms, amino, C₁-C₄-alkylamino and di-C₁-C₄-alkylamino;
      and
R³ and R⁴ independently of one another represent hydrogen, fluorine, chlorine, bromine, cyano, nitro or represent C₁-C₆-alkyl, C₂-C₆-alkenyl or C₂-C₆-alkynyl which are in each case optionally mono- to tetrasubstituted by identical or different substituents from the group consisting of fluorine, chlorine, bromine, nitro, cyano, phenyl, C₁-C₄-alkoxy, C₁-C₄-haloalkoxy having 1 to 7 identical or different fluorine, chlorine or bromine atoms, C₁-C₄-alkylthio, C₁-C₄-haloalkylthio having 1 to 7 identical or different fluorine, chlorine or bromine atoms, C₁-C₄-acyl, C₁-C₄-acyloxy, C₁-C₄-alkoxy-carbonyl, amino, C₁-C₄-alkylamino, di-C₁-C₄-alkylamino, phenylamino and diphenylamino;
   or represent phenyl which is optionally mono- to tetrasubstituted by identical or different substituents from the group consisting of fluorine, chlorine, bromine, cyano, nitro, C₁-C₄-alkyl, C₁-C₄-haloalkyl having 1 to 4 identical or different fluorine, chlorine or bromine atoms, C₁-C₄-alkoxy, C₁-C₄-haloalkoxy having 1 to 4 identical or different fluorine, chlorine or bromine atoms, C₁-C₄-alkylthio, C₁-C₄-haloalkylthio having 1 to 4 identical or different fluorine, chlorine or bromine atoms, amino, C₁-C₄-alkylamino and di-C₁-C₄-alkylamino;
   or represent heterocyclyl which is optionally mono- to tetrasubstituted by identical or different substituents from the group consisting of fluorine, chlorine, bromine, cyano, nitro, C₁-C₄-alkyl, C₁-C₄-haloalkyl having 1 to 4 identical or different fluorine, chlorine or bromine atoms, C₁-C₄-alkoxy, C₁-C₄-haloalkoxy having 1 to 4 identical or different fluorine, chlorine or bromine atoms, C₁-C₄-alkylthio, C₁-C₄-haloalkylthio having 1 to 4 identical or different fluorine, chlorine or bromine atoms, amino, C₁-C₄-alkylamino and di-C₁-C₄-alkylamino.

Very particular preference is given to compounds of the formula (I) in which
R¹ and R² independently of one another represent hydrogen, or represent C₁-C₄-alkyl, C₂-C₄-alkenyl or C₂-C₄-alkynyl which are in each case optionally mono- to trisubstituted by identical or different substituents from the group consisting of fluorine, chlorine, bromine, nitro, cyano and phenyl,
   or represent phenyl which is optionally mono- to disubstituted by identical or different substituents from the group consisting of fluorine, chlorine, bromine, cyano, nitro, C₁-C₂-alkyl, halomethyl having 1 to 3 identical or different fluorine or chlorine atoms, amino, monomethylamino and dimethylamino,
   or represent heterocyclyl which is optionally mono- to disubstituted by identical or different substituents from the group consisting of fluorine, chlorine, bromine, cyano, nitro, C₁-C₂-alkyl, C₁-haloalkyl having 1 to 3 identical or different fluorine or chlorine atoms, amino, monomethylamino and dimethylamino,
   or represent —COR⁵, —CONR⁶, —CSNR⁷ or —SO₂R⁸ stehen,
      where
R⁵ to R⁸ independently of one another represent hydrogen or represent C₁-C₅-alkyl, C₂-C₅-alkenyl or C₂-C₅-alkynyl which are in each case optionally mono- to trisubstituted by identical or different substituents from the group consisting of fluorine, chlorine, bromine, nitro, cyano and phenyl;
   or represent phenyl which is optionally mono- to disubstituted by identical or different substituents from the group consisting of fluorine, chlorine, bromine, cyano, nitro, C₁-C₂-alkyl, halomethyl having 1 to 3 identical or different fluorine or chlorine atoms, methoxy, amino, monomethylamino and dimethylamino;
   or represent heterocyclyl which is optionally mono- to disubstituted by identical or different substituents from the group consisting of fluorine, chlorine, bromine, cyano, nitro, C₁-C₂-alkyl, halomethyl having 1 to 3 identical or different fluorine or chlorine atoms, amino, monomethylamino and dimethylamino;
      and
R³ and R⁴ independently of one another represent hydrogen or represent C₁-C₅-alkyl, C₂-C₅-alkenyl or C₂-C₅-alkynyl which are in each case optionally mono- to trisubstituted by identical or different substituents from the group consisting of fluorine, chlorine, bromine, nitro, cyano and phenyl;
   or represent phenyl which is optionally mono- to disubstituted by identical or different substituents from the group consisting of fluorine, chlorine, bromine, cyano, nitro, methoxy C₁-C₂-alkyl, halomethyl having 1 to 3 identical or different fluorine or chlorine atoms, amino, monomethylamino and dimethylamino;
   or represent heterocyclyl which is optionally mono- to disubstituted by identical or different substituents from the group consisting of fluorine, chlorine, bromine, cyano, nitro, C₁-C₂-alkyl, halomethyl having 1 to 3 identical or different fluorine or chlorine atoms, amino, monomethylamino and dimethylamino.

Especially preferred are compounds of the formula (I) in which
R¹ and R² independently of one another represent hydrogen or represent C₁-C₅-alkyl,
   or represent —COR⁵, —CONR⁶, —CSNR⁷ or —SO₂R⁸,
      where
R⁵ represents C₁-C₅-alkyl which is optionally mono- to trisubstituted by identical or different substituents from the group consisting of fluorine and chlorine,
   or represents phenyl which is optionally substituted by fluorine, chlorine, methyl, halomethyl having 1 to 3 identical or different substituents from the group consisting of fluorine and chlorine atoms,
   or represents 2-furyl or 2-thienyl which are in each case optionally substituted by methyl, fluorine or chlorine;
      where
R₆ represents phenyl which is optionally mono- to disubstituted by fluorine, chlorine, methyl, halomethyl having 1 to 3 identical or different substituents from the group consisting of fluorine and chlorine atoms,
R⁷ represents C₁-C₅-alkyl or represents phenyl which is optionally substituted by fluorine, chlorine, methyl, halomethyl having 1 to 3 identical or different fluorine or chlorine atoms;
R⁸ represents C₁-C₅-alkyl which is optionally mono- to trisubstituted by identical or different substituents from the group consisting of fluorine and chlorine, or represents phenyl which is optionally substituted by fluorine, chlorine, methyl, methoxy, halomethyl having 1 to 3 identical or different substituents from the group consisting of fluorine and chlorine atoms, or represents 2-furyl or 2-thienyl which are in each case optionally substituted by methyl, fluorine or chlorine;
   and
R³ and R⁴ independently of one another represent hydrogen or represent C₁-C₅-alkyl which is optionally mono- to trisubstituted by identical or different substituents from the group consisting of fluorine and chlorine,
   or represent phenyl which is optionally mono- to disubstituted by fluorine, chlorine, methyl, methoxy, halomethyl having 1 to 3 identical or different substituents from the group consisting of fluorine and chlorine atoms, or represent furyl, thienyl, pyridyl, which are in each case optionally mono- to disubstituted by identical or different substituents from the group consisting of fluorine, chlorine, methyl, halomethyl having 1 to 3 identical or different substituents from the group consisting of fluorine and chlorine atoms.

The radical definitions given in the respective combinations of preferred and particularly preferred and especially preferred combinations of radicals specifically for these radicals can, independently of the combination given in each case, also be replaced by any radical definitions of other combinations. Moreover, it is also possible for radical definitions from any of the preferred ranges not to apply.

The compound of the formula (I)

in which

A represents a radical 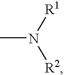
   and
R¹ and R² represent hydrogen,
   can be prepared by reacting hydroxylamine or its salts with 2-amino-1-cyclopentene-1-carbonitrile, if appropriate in the presence of diluents and if appropriate in the presence of a catalytic or stoichiometric amount of base.

Suitable diluents which are added, if appropriate, are both water and all customary organic solvents. These preferably include alcohols, such as ethanol or propanol, hydrocarbons, such as toluene, xylene or hexane, chlorinated hydrocarbons, such as chlorobenzene, methylene chloride or chloroform, ketones, such as acetone or butanone, ethers, such as tetrahydrofuran, diethyl ether, methyl tert-butyl ether, dimethoxyethane or dioxane, nitrites, such as acetonitrile, amides such as N,N-dimethylformamide, N,N-dimethylacetamide or N-methylpyrrolidone, sulfoxides, such as dimethyl sulfoxide, sulfones, such as sulfolane, and also esters, such as ethyl acetate or methyl acetate.

In the preparation process, the reaction temperature can be varied within a wide temperature range. In general, the process is carried out between −30° C. and +150° C., preferably between 0° C. and +110° C.

The preparation of 2-amino-1-cyclopentene-1-carbonitrile is already known from the literature (cf. Q. E. Thompson, J. Am. Chem. Soc. 1958, 80, 5483-5487).

The compounds of the formula (I)

in which

A represents a radical 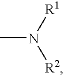
   and
R¹ and R² independently of one another represent hydrogen, halogen, cyano, nitro or represent in each case optionally substituted alkyl, alkenyl, alkynyl, aryl, heterocycyl, —COR⁵, —CONR⁶, —CSNR⁷ or —SO₂R⁸,
   where
R⁵ to R⁸ independently of one another represent in each case optionally substituted alkyl, alkenyl, alkynyl, aryl and heterocyclyl,
   and,
R³ and R⁴ independently of one another represent hydrogen, or represent in each case optionally substituted alkyl, alkenyl, alkynyl, aryl and heterocyclyl,
   can be prepared by reacting the compound of the formula (I)
   in which
A represents a radical 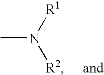
R¹ and R² represent hydrogen
a) with carboxylic anhydrides of the formula (II) 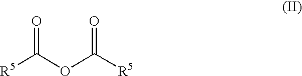
   in which R⁵ is as defined above,
   if appropriate in the presence of diluents and if appropriate in the presence of a catalytic or stoichiometric amount of base;
      or
b) with carbonyl halides of the formula (III) 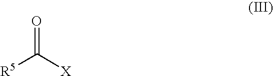
   in which R⁵ is as defined above and X represents Cl and Br,
   if appropriate in the presence of diluents and if appropriate in the presence of a catalytic or stoichiometric amount of base;
      or
c) with isocyanates of the formula (IV) 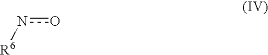
   in which R⁶ is as defined above,
   if appropriate in the presence of diluents and if appropriate in the presence of a catalytic or stoichiometric amount of base;
      or
d) with isothiocyanates of the formula (V) 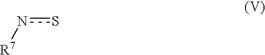
   in which R⁷ is as defined above,
   if appropriate in the presence of diluents and if appropriate in the presence of a catalytic or stoichiometric amount of base;
      or
e) with sulfonyl chlorides of the formula (VI) 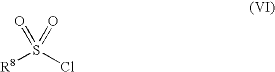
   in which R⁸ is as defined above,
   if appropriate in the presence of diluents and if appropriate in the presence of a catalytic or stoichiometric amount of base.

Suitable diluents which are added, if appropriate, are both water and all customary organic solvents. These preferably include alcohols, such as ethanol or propanol, hydrocarbons, such as toluene, xylene or hexane, chlorinated hydrocarbons, such as chlorobenzene, methylene chloride or chloroform, ketones, such as acetone or butanone, ethers, such as tetrahydrofuran, diethyl ether, methyl tert-butyl ether, dimethoxyethane or dioxane, nitriles, such as acetonitrile, amides such as N,N-dimethylformamide, N,N-dimethylacetamide or N-methylpyrrolidone, sulfoxides, such as dimethyl sulfoxide, sulfones, such as sulfolane, and also esters, such as ethyl acetate or methyl acetate.

In the preparation process, the reaction temperature can be varied within a wide temperature range. In general, the process is carried out between −30° C. and +150° C., preferably between 0° C. and +110° C.

The compounds of the formula (I)

in which

A represents 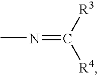
   in which R³ and R⁴ are as defined above,
   can be prepared by reacting the compound of the formula (1)
   in which
A represents 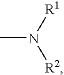
and R¹ and R² represent hydrogen
   with aldehydes or ketones of the formula (VII) 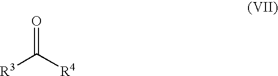
   in which R³ and R⁴ are as defined above,
   if appropriate in the presence of diluents and if appropriate in the presence of a catalytic or stoichiometric amount of base.

Suitable diluents which are added, if appropriate, are both water and all customary organic solvents. These preferably include alcohols, such as ethanol or propanol, hydrocarbons, such as toluene, xylene or hexane, chlorinated hydrocarbons, such as chlorobenzene, methylene chloride or chloroform, ketones, such as acetone or butanone, ethers, such as tetrahydrofuran, diethyl ether, methyl tert-butyl ether, dimethoxyethane or dioxane, nitrites, such as acetonitrile, amides such as N,N-dimethylformamide, N,N-dimethylacetamide or N-methylpyrrolidone, sulfoxides, such as dimethyl sulfoxide, sulfones, such as sulfolane, and also esters, such as ethyl acetate or methyl acetate.

In the preparation process, the reaction temperature can be varied within a wide temperature range. In general, the process is carried out between −30° C. and +150° C., preferably between 0° C. and +110° C.

The compounds of the formulae (I) and (II) according to the invention have potent microbicidal action and can be used for controlling unwanted microorganisms, such as fungi and bacteria, in crop protection and in the protection of materials.

In the protection of materials, the substances according to the invention can be used for protecting industrial materials against attack and destruction by undesirable microorganisms. In the present context, industrial materials are to be understood as meaning non-living materials which have been prepared for use in industry. For example, industrial materials which are intended to be protected by active compounds according to the invention from microbial change or destruction can be glues, sizes, paper and board, textiles, leather, wood, paints and synthetic articles, cooling lubricants and other materials which can be attacked or destroyed by microorganisms. Parts of production plants, for example cooling-water circuits, which may be impaired by the multiplication of microorganisms may also be mentioned in the context of the materials to be protected. Industrial materials which may preferably be mentioned in the context of the present invention are glues, sizes, paper and boards, leather, wood, paints, cooling lubricants and heat transfer liquids.

Examples of microorganisms which are capable of bringing about degradation of, or change in, the industrial materials and which may be mentioned are bacteria, fungi, yeasts, algae and slime organisms. The active compounds according to the invention preferably act against fungi, in particular moulds, wood-discolouring and wood-destroying fungi (Basiidiomycetes) and also against slime organisms and bacteria.

Microorganisms of the following genera may be mentioned by way of example:

Altemaria, such as Altemaria tenuis,

Aspergillus, such as Aspergillus niger,

Chaetomium, such as Chaetomium globosum,

Coniophora, such as Coniophora puetana,

Lentinus, such as Lentinus tigrinus,

Penicillium, such as Penicillium glaucum,

Polyporus, such as Polyporus versicolor,

Aureobasidium, such as Aureobasidium pullulans,

Sclerophoma, such as Sclerophoma pityophila,

Trichoderma, such as Trichoderma viride,

Escherichia, such as Escherichia coli,

Pseudomonas, such as Pseudomonas aeruginosa,

Staphylococcus, such as Staphylococcus aureus.

The compounds (I) according to the invention can, on their own or in any mixture with one another, be used for protecting industrial materials.

Depending on their respective physical and/or chemical properties, the compounds according to the invention or mixtures thereof can be converted into customary formulations, such as solutions, emulsions, suspensions, powders, foams, pastes, granules, aerosols and very fine capsules in polymeric substances.

These formulations and compositions are prepared in a known manner, for example by mixing the active compounds with extenders, that is, liquid solvents, liquefied gases under pressure, and/or solid carriers, if appropriate with the use of surfactants, that is emulsifiers and/or dispersants and/or foam-formers. If the extender used is water, it is also possible to use for example organic solvents as auxiliary solvents. Essentially, suitable liquid solvents are: aromatics, such as xylene, toluene or alkylnaphthalenes, chlorinated aromatics or chlorinated aliphatic hydrocarbons, such as chlorobenzenes, chloroethylene or methylene chloride, aliphatic hydrocarbons, such as cyclohexane or paraffins, for example mineral oil fractions, alcohols, such as butanol or glycol and their ethers and esters, ketones, such as acetone, methyl ethyl ketone, methyl isobutyl ketone or cyclohexanone, strongly polar solvents, such as dimethyl formamide and dimethyl sulfoxide, and water. By liquefied gaseous extenders or carriers are meant liquids which are gaseous at ambient temperature and under atmospheric pressure, for example aerosol propellants, such as halogenated hydrocarbons and butane, propane, nitrogen and carbon dioxide. Suitable solid carriers are: for example ground natural minerals, such as kaolins, clays, talc, chalk, quartz, attapulgite, montmorillonite or diatomaceous earth, and ground synthetic minerals, such as finely divided silica, alumina and silicates. Suitable solid carriers for granules are: for example crushed and fractionated natural rocks such as calcite, marble, pumice, sepiolite and dolomite, and synthetic granules of organic and inorganic meals, and granules of organic material such as sawdust, coconut shells, maize hobs and tobacco stalks. Suitable emulsifiers and/or foam-formers are: for example nonionic and anionic emulsifiers, such as polyoxyethylene fatty acid esters, polyoxyethylene fatty alcohol ethers, for example alkylaryl polyglycol ethers, alkylsulfonates, alkyl sulfates, arylsulfonates and protein hydrolysates. Suitable dispersants are: for example lignin-sulfite waste liquors and methylcellulose.

Tackifiers such as carboxymethylcellulose and natural and synthetic polymers in the form of powders, granules or latices, such as gum arabic, polyvinyl alcohol and polyvinyl acetate, and natural phospholipids, such as cephalins and lecithins, and synthetic phospholipids, can be used in the formulations. Possible further additives are mineral and vegetable oils.

It is possible to use colorants such as inorganic pigments, for example iron oxide, titanium oxide and Prussian blue, and organic dyestuffs, such as alizarin dyestuffs, azo dyestuffs and metal phthalocyanin dyestuffs, and trace nutrients such as salts of iron, manganese, boron, copper, cobalt, molybdenum and zinc.

The formulations generally comprise between 0.1 and 95% by weight of active compound or active compound mixture, preferably between 2 and 75% by weight.

The present invention furthermore provides microbicidal compositions based on the compounds according to the invention, which compositions comprise at least one solvent or diluent and, if appropriate, processing auxiliaries and, if appropriate, further antimicrobially active substances.

The efficacy and the activity spectrum of the active compounds of the formulae (I) and (II) and of the compositions preparable therefrom, of precursors or of formulations in general can be increased by adding, if appropriate, further antimicrobial compounds, fungicides, bactericides, herbicides, insecticides or other active compounds, so as to widen the spectrum of activity or to obtain particular effects such as, for example, additional protection against insects. These mixtures may have a wider activity spectrum than the compounds according to the invention.

In many cases, synergistic effects are obtained, i.e. the activity of the mixture is greater than the activity of the individual components. The following co-components are found to be particularly favorable:

triazoles such as:

azaconazole, azocyclotin, bitertanol, bromuconazole, cyproconazole, diclobutrazole, difenoconazole, diniconazole, epoxyconazole, etaconazole, fenbuconazole, fenchlorazole, fenethanil, fluquinconazole, flusilazole, flutriafol, furconazole, hexaconazole, imibenconazole, ipconazole, isozofos, myclobutanil, metconazole, paclobutrazol, penconazole, propioconazole, prothioconazole, simeoconazole, (±)-cis-1-(4-chlorophenyl)-2-(1H-1,2,4-triazol-1-yl)-cycloheptanol, 2-(1-tert-butyl)-1-(2-chlorophenyl)-3-(1,2,4-triazol-1-yl)-propan-2-ol, tebuconazole, tetraconazole, triadimefon, triadimenol, triapenthenol, triflumizole, triticonazole, uniconazole and their metal salts and acid adducts;

imidazoles such as:

clotrimazole, bifonazole, climbazole, econazole, fenapamil, imazalil, isoconazole, ketoconazole, lombazole, miconazole, pefurazoate, prochloraz, triflumizole, thiazolcar, 1-imidazolyl-1-(4′-chlorophenoxy)-3,3-dimethylbutan-2-one, and their metal salts and acid adducts;

pyridines and pyrimidines such as:

ancymidol, buthiobate, fenarimol, mepanipyrin, nuarimol, pyvoxyfur, triamirol;

succinate dehydrogenase inhibitors such as:

benodanil, carboxim, carboxim sulfoxide, cyclafluramid, fenfuram, flutanil, furcarbanil, furmecyclox, mebenil, mepronil, methfuroxam, metsulfovax, pyrocarbolid, nicobifen, oxycarboxin, shirlan, Seedvax;

naphthalene derivatives such as:

terbinafine, naftifine, butenafine, 3-chloro-7-(2-aza-2,7,7-trimethyl-oct-3-en-5-yne);

sulfenamides such as:

dichlofluanid, tolylfluanid, folpet, fluorofolpet, captan, captofol;

Benzimidazoles such as:

carbendazim, benomyl, fuberidazole, thiabendazole or their salts;

morpholine derivatives such as:

aldimorph, dimethomorph, dodemorph, falimorph, fenpropidin fenpropimorph, tridemorph, trimorphamid and their arylsulfonate salts such as, for example, p-toluenesulfonic acid and p-dodecylphenyl sulfonic acid;

benzothiazoles such as:

2-mercaptobenzothiazole;

benzothiophene dioxides such as:

N-cyclohexyl-benzo[b]thiophene carboxamide;

benzamides such as:

2,6-dichloro-N-(4-trifluoromethylbenzyl)-benzamide, tecloftalam;

boron compounds such as:

boric acid, boric ester, borax;

formaldehyde and formaldehyde-releasing compounds such as:

benzyl alcohol mono-(poly)-hemiformal, n-butanol hemiformal, dazomet, ethylene glycol hemiformal, hexa-hydro-5-triazine, hexamethylenetetramine, N-hydroxymethyl-N′-methylthiourea, N-methylolchloroacetamide, oxazolidine, paraformaldehyde, taurolin, tetrahydro-1,3-oxazine, N-(2-hydroxypropyl)-amine-methanol, tetramethyloylacetylenediurea;

isothiazolinones such as:

N-methylisothiazolin-3-one, 5-chloro-N-methylisothiazolin-3-one, 4,5-dichloro-N-octylisothiazolin-3-one, 5-chloro-N-octylisothiazolinone, N-octyl-isothiazolin-3-one, 4,5-trimethylene-isothiazolinone, 4,5-benzoisothiazolinone;

aldehydes such as:

cinnamaldehyde, formaldehyde, glutardialdehyde, β-bromocinnamaldehyde, o-phthaldialdehyde;

thiocyanates such as:

thiocyanatomethylthiobenzothiazole, methylenebisthiocyanate;

quaternary ammonium compounds and guanidine such as:

benzalkonium chloride, benzyldimethyltetradecylammonium chloride, benzyldimethyldodecylammonium chloride, dichlorobenzyldimethylalkylammonium chloride, didecyldimethylammonium chloride, dioctyldimethylammonium chloride, N-hexadecyltrimethylammonium chloride, 1-hexadecylpyridinium chloride, iminoctadine tris (albesilate);

iodine derivatives such as:

diiodomethyl p-tolyl sulfone, 3-iodo-2-propynyl alcohol, 4-chlorophenyl-3-iodo-propargylformal, 3-bromo-2,3-diiodo-2-propenyl ethylcarbamate, 2,3,3-triiodoallyl alcohol, 3-bromo-2,3-diiodo-2-propenyl alcohol, 3-iodo-2-propynyl n-butylcarbamate, 3-iodo-2-propynyl n-hexylcarbamate, 3-iodo-2-propynyl-cyclohexylcarbamate, 3-iodo-2-propynyl phenylcarbamate;

phenols such as:

tribromophenol, tetrachlorophenol, 3-methyl-4-chlorophenol, 3,5-dimethyl-4-chlorophenol, dichlorophene, 2-benzyl-4-chlorophenol, triclosan, diclosan, hexachlorophene, p-hydroxybenzoate, o-phenylphenol, m-phenylphenol, p-phenylphenol 4-(2-tert-butyl-4-methylphenoxy)phenol, 4-(2-isopropyl-4-methylphenoxy (phenol,4-(2,4-dimethylphenoxy)phenol and their alkali metal salts and alkaline earth metal salts;

microbicides with an activated halogen group such as:

bronopol, bronidox, 2-bromo-2-nitro-1,3-propanediol, 2-bromo-4′-hydroxy-acetophenone, 1-bromo-3-chloro-4,4,5,5-tetramethyl-2-imidazolidinone, β-brom-β-nitrostyrene, chloracetamide, chloramine T, 1,3-dibromo-4,4,5,5-tetramethyl-2-imidazolidinone, dichloramine T, 3,4-dichloro-(3H)-1,2-dithiol-3-one, 2,2dibromo-3-nitrile-propionamide, 1,2-dibromo-2,4-dicyanobutane, halane, halazone, mucochloric acid, phenyl (2-chlorocyano-vinyl) sulfone, phenyl (1,2-dichloro-2-cyanovinyl) sulfone, trichloroisocyanuric acid;

pyridines such as:

1-hydroxy-2-pyridinethione (and their Cu, Na, Fe, Mn, Zn salts), tetrachloro-4-methylsulfonylpyridine, pyrimethanol, mepanipyrim, dipyrithion, 1-hydroxy-4-methyl-6-(2,4,4-trimethylpentyl)-2(1H)-pyridine;

methoxyacrylates or similar such as:

azoxystrobin, dimoxystrobin, fluoxastrobin, kresoxim-methyl, metominostrobin, orysastrobin, picoxystrobin, pyraclostrobin, trifloxystrobin, 2,4-dihydro-5-methoxy-2-methyl-4-[2-[[[[1-[3-(trifluoromethyl)phenyl]ethylidene]amino]oxy]-methyl]phenyl]-3H-1,2,4-triazol-3-one (CAS-No. 185336-79-2);

metal soaps such as:

tin naphthenate, tin octoate, tin 2-ethylhexanoate, tin oleate, tin phosphate, tin benzoate, copper naphthenate, copper octoate, copper 2-ethylhexanoate, copper oleate, copper phosphate, copper benzoate, zinc naphthenate, zinc octoate, zinc 2-ethylhexanoate, zinc oleate, zinc phosphate, zinc benzoate;

metal salts such as:

copper hydroxycarbonate, sodium dichromate, potassium dichromate, potassium chromate, copper sulfate, copper chloride, copper borate, zinc fluorosilicate, copper fluorosilicate;

oxides such as:

oxides of the metals tin, copper and zinc, such as, for example, tributyltin oxide, Cu₂O, CuO, ZnO;

oxidizing agents such as:

hydrogen peroxide, peracetic acid, potassium persulfate;

dithiocarbamates such as:

cufraneb, ferban, potassium N-hydroxymethyl-N′-methyldithiobarbamate, sodium dimethyldithiocarbamate, potassium dimethyldithiocarbamate, macozeb, maneb, metam, metiram, thiram, zineb, ziram;

nitriles such as:

2,4,5,6-tetrachloroisophthalonitrile, disodium cyano-dithioimidocarbamate;

quinolines such as:

8-hydroxyquinoline and their copper salts;

other fungicides and bactericides such as:

bethozaxin, 5-hydroxy-2(5H)-furanone, 4,5-benzodithiazolinone, 4,5-trimethylenedithiazolinone, N-(2-p-chlorobenzoylethyl)-hexaminium chloride, 2-oxo-2-(4-hydroxyphenyl)acetohydroxycinnamoyl chloride, tris-N-(cyclohexyldiazeniumdioxy)-aluminum, N-(cyclo-hexyldiazeniumdioxy)-tributyltin or its potassium salts, bis-N-(cyclohexyldiazeniumdioxy)-copper; iprovalicarb, fenhexamide, spiroxamine, carpropamid, diflumetorin, quinoxyfen, famoxadone, polyoxorim, acibenzolar S-methyl, furametpyr, thifluzamide, methalaxy-M, benthiavalicarb, metrafenon, cyflufenamid, tiadinil, tea tree oil, phenoxyethanol,

Ag, Zn or Cu-containing zeolites alone or incorporated into polymeric materials.

Very especially preferred are mixtures with

azaconazole, bromuconazole, cyproconazole, dichlobutrazol, diniconazole, hexaconazole, metaconazole, penconazole, propiconazole, tebuconazole, dichlofluanid, tolylfluanid, fluorfolpet, methfuroxam, carboxin, benzo[b]thiophene S,S-dioxide cyclohexylcarboxamide, fenpiclonil, 4-(2,2-difluoro-1,3-benzodioxol-4-yl)-1H-pyrrole-3-carbonitrile, butenafine, imazalil, N-methyl-isothiazolin-3-one, 5-chloro-N-methylisothiazolin-3-one, N-octylisothiazolin-3-one, dichloro-N-octylisothiazolinone, mercaptobenthiazole, thiocyanatomethylthiobenzothiazole, benzoisothiazolinone, N-(2-hydroxypropyl)-amino-methanol, benzyl alcohol (hemi)-formal, N-methylolchloroacetamide, N-(2-hydroxypropyl)-amine-methanol, glutaraldehyde, omadine, dimethyl dicarbonate, 2-bromo-2-nitro-1,3-propanediol and/or 3-iodo-2-propinyl n-butylcarbamate, bethoxazin, o-phthaldialdehyde.

Apart from with the abovementioned fungicides and bactericides, mixtures with a good efficacy are, moreover, also prepared with other active compounds:

insecticides/acaricides/nematicides such as:

abamectin, acephate, acetamiprid, acetoprole, acrinathrin, alanycarb, aldicarb, aldoxycarb, aldrin, allethrin, alpha-cypermethrin amidoflumet, amitraz, avermectin, azadirachtin, azinphos A, azinphos M, azocyclotin,

Bacillus thuringiensis, barthrin, 4-bromo-2(4-chlorophenyl)-1-(ethoxymethyl)-5-(trifluoromethyl)-1H-pyrrole-3-carbonitrile, bendiocarb, benfuracarb, bensultap, betacyfluthrin, bifenthrin, bioresmethrin, bioallethrin, bistrilfluron, bromophos A, bromophos M, bufencarb, buprofezin, butathiophos, butocarboxim, butoxycarboxim,

cadusafos, carbaryl, carbofuran, carbophenothion, carbosulfan, cartap, quinomethionate, cloethocarb, 4-chloro-2-(2-chloro-2-methylpropyl)-5-[(6-iodo-3-pyridinyl)methoxy]-3(2H)-pyridazinone (CAS-RN: 120955-77-3), chlordane, chlorethoxyfos, chlorfenapyr, chlorfenvinphos, chlorfluazuron, chlormephos, N-[(6-chloro-3-pyridinyl)-methyl]-N′-cyano-N-methyl-ethaneimidamide, chlorpicrin, chlorpyrifos A, chlorpyrifos M, cis-resmethrin, clocythrin, cypophenothrin clofentezin, coumaphos, cyanophos, cycloprothrin, cyfluthrin, cyhalothrin, cyhexatin, cypermethrin, cyromazin,

decamethrin, deltamethrin, demeton M, demeton S, demeton-S-methyl, diafenthiuron, dialiphos, diazinon, 1,2-dibenzoyl-1(1,1-dimethyl)-hydrazine, DNOC, dichlofenthion, dichlorvos, dicliphos, dicrotophos, difethialone, diflubenzuron, dimethoate, 3,5-dimethylphenyl methylcarbamate, dimethyl-(phenyl)-silyl-methyl-3-phenoxybenzyl ether, dimethyl-(4-ethoxyphenyl)-silylmethyl-3-phenoxybenzyl ether, dimethylvinphos, dioxathion, disulfoton, eflusilanate, emamectin, empenthrin, endosulfan, EPN, esfenvalerate, ethiofencarb, ethion, ethofenprox, etrimphos, etoxazole, etobenzanid, fenamiphos, fenazaquin, fenbutatin oxide, fenfluthrin, fenitrothion, fenobucarb, fenothiocarb, fenoxycarb, fenpropathrin, fenpyrad, fenpyroximat, fensulfothion, fenthion, fenvalerate, fipronil, flonicamid, fluacrypyrim, fluazuron, flucycloxuron, flucythrinate, flufenerim, flufenoxuron, flupyrazotos, flufenzine, flumethrin, flufenprox, fluvalinate, fonophos, formethanate, formothion, fosmethilan fosthiazate, fubfenprox, furathiocarb,

halofenocid, HCH, (CAS RN: 58-89-9), heptenophos, hexaflumuron, hexythiazox, hydramethylnon, hydroprene,

imidacloprid, imiprothrin, indoxycarb, iodfenfos, iprinomectin, iprobenfos, isazophos, isoamidophos, isofenphos, isoprocarb, isoprothiolane, isoxathion, ivermectin,

kadedrin

lambda-cyhalothrin, lufenuron,

malathion, mecarbam, mervinphos, mesulfenphos, metaldehyde, methacrifos, methamidophos, methidathion, methiocarb, methomyl, metalcarb, milbemectin, monocrotophos, moxiectin,

naled, NI 125, nicotine, nitenpyram, noviflumuron

omethoate, oxamyl, oxydemethon M, oxydeprofos,

parathion A, parathion M, penfluron, permethrin, 2-(4-phenoxyphenoxy)-ethyl ethylcarbamate, phenthoate, phorate, phosalon, phosmet, phosphamidon, phoxim, pirimicarb, pirimiphos M, pirimiphos A, prallethrin, profenophos, promecarb, propaphos, propoxur, prothiophos, prothoate, pymetrozin, pyrachlophos, pyridaphenthion, pyresmethrin, pyrethrum, pyridaben, pyridalyl pyrimidifen, pyriproxifen, pyrithiobac-sodium,

quinalphos,

resmethrin, rotenone,

salithion, sebufos, silafluofen, spinosad, spirodiclofen, spiromesifen, sulfotep, sulprofos,

tau-fluvalinate, taroils, tebufenozide, tebufenpyrad, tebupirimphos, teflubenzuron, tefluthrin, temephos, terbam, terbufos, tetrachlorvinphos, tetramethrin, Tetramethacarb, thiacloprid, thiafenox, thiamethoxam, thiapronil, thiodicarb, thiofanox, thiazophos, thiocyclam, thiomethon, thionazin, thuringiensin, tralomethrin, transfluthrin, triarathen, triazophos, triazamate, triazuron trichlorfon, triflumuron, trimethacarb,

vamidothion, xylylcarb, zetamethrin;

molluscicides:

fentin acetate, metaldehyde, methiocarb, niclosamide;

herbicides and algicides

acetochlor, acifluorfen, aclonifen, acrolein, alachlor, alloxydim, ametryn, amidosulfuron, amitrole, ammonium sulfamate, anilofos, asulam, atrazine, azafenidin, aziptrotryne, azimsulfuron,

benazolin, benfluralin, benfuresate, bensulfuron, bensulfide, bentazone, benzofencap, benzthiazuron, bifenox, bispyribac, bispyribac-sodium, borax, bromacil, bromobutide, bromofenoxim, bromoxynil, butachlor, butamifos, butralin, butylate, bialaphos, benzoyl-prop, bromobutide, butroxydim,

carbetamide, carfentrazone-ethyl, carfenstrole, chlomethoxyfen, chloramben, chlorbromuron, chlorflurenol, chloridazon, chlorimuron, chlomitrofen, chloroacetic acid, chloransulam-methyl, cinidon-ethyl, chlorotoluron, chloroxuron, chlorpropham, chlorsulfuron, chlorthal, chlorthiamid, cinmethylin, cinofulsuron, clefoxydim, clethodim, clomazone, chlomeprop, clopyralid, cyanamide, cyanazine, cycloate, cycloxydim, chloroxynil, clodinafop-propargyl, cumyluron, clometoxyfen, cyhalofop, cyhalofop-butyl, clopyrasuluron, cyclosulfamuron,

diclosulam, dichlorprop, dichlorprop-P, diclofop, diethatyl, difenoxuron, difenzoquat, diflufenican, diflufenzopyr, dimefuron, dimepiperate, dimethachlor, dimethipin, dinitramine, dinoseb, dinoseb acetate, dinoterb, diphenamid, dipropetryn, diquat, dithiopyr, diduron, DNOC, DSMA, 2,4-D, daimuron, dalapon, dazomet, 2,4-DB, desmedipham, desmetryn, dicamba, dichlobenil, dimethamid, dithiopyr, dimethametryn,

eglinazine, endothal, EPTC, esprocarb, ethalfluralin, ethidimuron, ethofumesate, ethobenzanid, ethoxyfen, ethametsulfuron, ethoxysulfuron,

fenoxaprop, fenoxaprop-P, fenuron, flamprop, flamprop-M, flazasulfuron, fluazifop, fluazifop-P, fuenachlor, fluchloralin, flufenacet, flumeturon, fluorocglycofen, fluoronitrofen, flupropanate, flurenol, fluridone, flurochloridone, fluroxypyr, fomesafen, fosamine, fosametine, flamprop-isopropyl, flamprop-isopropyl-L, flufenpyr flumiclorac-pentyl, flumipropyn, flumioxzim, flurtamone, flumioxzim, flupyrsulfuron-methyl, fluthiacet-methyl,

glyphosate, glufosinate-ammonium

haloxyfop, hexazinone,

imazamethabenz, isoproturon, isoxaben, isoxapyrifop, imazapyr, imazaquin, imazethapyr, ioxynil, isopropalin, imazosulfuron, imazomox; isoxaflutole, imazapic, ketospiradox, lactofen, lenacil, linuron,

MCPA, MCPA-hydrazide, MCPA-thioethyl, MCPB, mecoprop, mecoprop-P, mefenacet, mefluidide, mesosulfuron metam, metamifop-metamitron, metazachlor, methabenzthiazuron, methazole, methoroptryne, methyldymron, methyl isothiocyanate, metobromuron, metoxuron, metribuzin, metsulfuron, molinate, manolide, monolinuron, MSMA, metolachlor, metosulam, metobenzuron,

naproanilide, napropamide, naptalam, neburon, nicosulfuron, norflurazon, sodium chlorate,

oxadiazon, oxyfluorfen, oxysulfuron, orbencarb, oryzalin, oxadiargyl, propyzamide, prosulfocarb, pyrazolate, pyrazolsulfuron, pyrazoxyfen, pyribenzoxim, pyributicarb, pyridate, paraquat, pebulate, pendimethalin, pentachlorophenol, pentoxazone, pentanochlor, petroleum oils, phenmedipham, picloram, piperophos, pretilachlor, primisulfuron, prodiamine, profoxydim, prometryn, propachlor, propanil, propaquizafob, propazine, propham, propisochlor, pyriminobac-methyl, pelargonic acid, pyrithiobac, pyraflufen-ethyl, quimnerac, quinocloamine, quizalofop, quizalofop-P, quinchlorac,

rimsulfuron

sethoxydim, sifuron, simazine, simetryn, sulfosulfuron, sulfometuron, sulfentrazone, sulcotrione, sulfosate,

tar oils, TCA, TCA-sodium, tebutam, tebuthiuron, terbacil, terbumeton, terbuthylazine, terbutryn, thiazafluoron, thifensulfuron, thiobencarb, thiocarbazil, tralkoxydim, tri-allate, triasulfuron, tribenuron, triclopyr, tridiphane, trietazine, trifluoralin, tycor, thdiazimin, thiazopyr, triflusulfuron,

vernolate.

The weight ratios of the active compounds in these active compound combinations can be varied within relatively wide ranges.

Preferably, the active compound combinations comprise the active compound in an amount of from 0.1 to 99.9%, in particular from 1 to 75%, especially preferably from 5 to 50%, the remainder to 100% being one or more of the co-components mentioned above.

The microbicidal compositions or concentrates used for protecting the industrial materials comprise the active compound or the active compound combination in a concentration of 0.01 and 95% by weight, in particular from 0.1 to 60% by weight.

The use concentrations of the active compounds or active compound combinations to be used depend on the nature and the occurrence of the microorganisms to be controlled and on the composition of the material to be protected. The optimum rate of application can be determined by test series. In general, the use concentrations are in the range from 0.001 to 5% by weight, preferably from 0.05 to 1.0% by weight, based on the material to be protected.

With the active compounds or compositions according to the invention, it is possible to replace, in an advantageous manner, the microbicidal compositions available to date by more effective compositions. They have good stability and, in an advantageous manner, a broad activity spectrum.

### EXAMPLES

### Example 1

34.75 g (0.5 mol) of hydroxylamine hydrochloride were added to a solution of 54.07 g (0.5 mol) of 2-amino-1-cyclopentene-1-carbonitrile in 250 ml of ethanol, and the mixture was heated at the boil for one hour. The resulting precipitate was filtered off and the filtrate was concentrated and taken up in a mixture of 60 ml of isopropanol and 240 ml of water. The mixture was heated to 40° C., and at this temperature 0.05 mol of 50 percent strength aqueous sodium hydroxide solution was added dropwise. The mixture was then stirred at 60° C. for another half an hour and then cooled to 10° C., and the solid formed was filtered off. Washing with water and drying gives 51.4 g (82% of theory) of 5,6-dihydro-4H-cyclopenta[c]isoxazole-3-amine of the formula 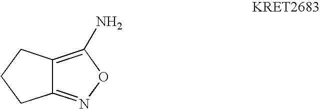
as a white solid, m.p.=135° C.

### Example 2

A mixture of 5.70 g (0.046 mol) of 5,6-dihydro-4H-cyclopenta[c]isoxazole-3-amine, 7.31 g (0.069 mol) of trimethyl orthoformate and a drop of conc. sulfuric acid was stirred at 110° C. for 2 hours and then heated at 170° C. for another 30 minutes. After cooling, 40 ml of 10 percent strength hydrochloric acid were added to the residue, and the mixture was heated at the boil for 3 hours. The mixture was then cooled to 0° C. and made alkaline (pH=12) using 20 percent strength aqueous sodium hydroxide solution. The resulting reaction mixture was then extracted with ethyl acetate and the extracts were subsequently concentrated under reduced pressure.

Subsequent column chromatography on silica gel (hexane/ethyl acetate=10/1) gave 0.13 g (2% of theory) of N-methyl-5,6-dihydro-4H-cyclopenta[c]isoxazole-3-amine of the formula 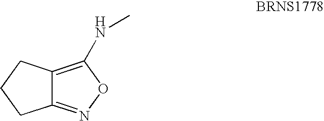
as a white solid, m.p.=52° C.

### Example 3

1.9 g (0.01 mol) of pivalic anhydride were added to a solution of 1.24 g (0.01 mol) of 5,6-dihydro-4H-cyclopenta[c]isoxazole-3-amine in 30 ml of toluene, and the mixture was stirred at 80° C. for 18 hours. The solvent was removed under reduced pressure, and subsequent column chromatography on silica gel (toluene/ethyl acetate=10/1) gave 0.28 g (13% of theory) of N-(5,6-dihydro-4H-cyclopenta[c]isoxazol-3-yl)-2,2-dimethylpropanamide of the formula 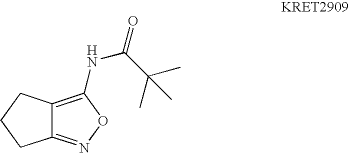
as a slightly yellowish solid, m.p.=113° C.

### Example 4

1.86 g (0.01 mol) of valeric anhydride were added to a solution of 1.24 g (0.01 mol) of 5,6-dihydro-4H-cyclopenta[c]isoxazole-3-amine in 30 ml of toluene, and the mixture was stirred at 80° C. for 14 hours. The solvent was removed under reduced pressure, and subsequent column chromatography on silica gel (toluene/ethyl acetate=10/1) gave 1.34 g (46% of theory) of N-(5,6-dihydro-4H-cyclopenta[c]isoxazol-3-yl)-N-pentanoylpentanamide of the formula 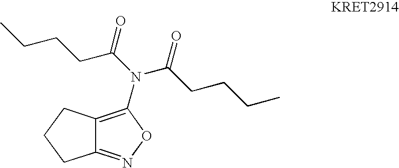
as a yellow oil, n_{D}²⁶=1.4760.

### Example 5

1.19 g (0.01 mol) of phenyl isocyanate and 0.12 g (0.001 mol) of DMAP were added to a solution of 1.24 g (0.01 mol) of 5,6-dihydro-4H-cyclopenta[c]isoxazole-3-amine in 25 ml of THF, and the mixture was stirred at 66° C. for 5 hours. The solvent was removed under reduced pressure, and subsequent column-chromatographic work-up on silica gel (toluene/ethyl acetate=10/1) gave 0.60 g (23% of theory) of N-(5,6-dihydro-4H-cyclopenta[c]isoxazol-3-yl)-N′-phenylurea of the formula 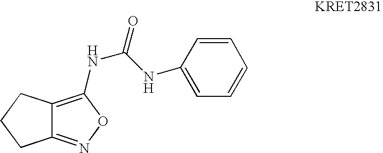
as a light-yellow solid, m.p.=202° C.

### Example 6

1.29 g (0.01 mol) of butyl isocyanate were added to a solution of 1.24 g (0.01 mol) of 5,6-dihydro-4H-cyclopenta[c]isoxazole-3-amine in 25 ml of THF, and the mixture was stirred at 66° C. for 17 hours. The solvent was removed under reduced pressure, and subsequent column chromatography on silica gel (toluene/ethyl acetate=8/1) gave 0.20 g (4% of theory) of N-butyl-N′-(5,6-dihydro-4H-cyclopenta[c]isoxazol-3-yl)thiourea of the formula 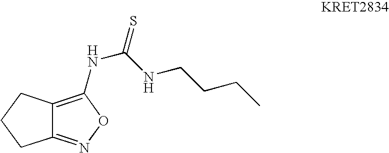
as a light-yellow solid, m.p.=93° C.

### Example 7

At 4° C., 0.23 g (0.006 mol) of sodium hydride (60% in oil) was initially charged in 15 ml of THF, and 0.70 g (0.006 mol) of 5,6-dihydro-4H-cyclopenta[c]isoxazole-3-amine was then added. The mixture was then stirred at 0° C. for 5 minutes, and 1.00 g (0.006 mol) of benzenesulfonyl chloride was then added. After 4 hours at room temperature, the solvent was removed under reduced pressure, and the residue was then chromatographed on silica gel (toluene/ethyl acetate=10/1). This gave 0.25 g (16% of theory) of N-(5,6-dihydro-4H-cyclopenta[c]isoxazol-3-yl)benzenesulfonamide of the formula 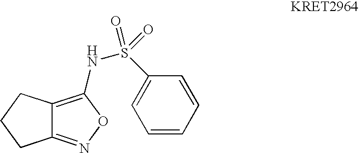
as a white solid, m.p.=132° C.

### Example 8

At 0° C., 0.32 g (0.008 mol) of sodium hydride (60% in oil) was initially charged in 20 ml of DMF, and 1.00 g (0.008 mol) of 5,6-dihydro-4H-cyclopenta[c]isoxazole-3-amine was then added. The mixture was stirred at 0° C. for 5 minutes, and 0.86 g (0.008 mol) of benzaldehyde was then added. After 3 hours at room temperature, the solvent was removed under reduced pressure, and the residue was then chromatographed on silica gel (toluene/ethyl acetate=10/1). Crystallization of the main fraction from toluene gives 0.13 g (7% of theory) of N-(5,6-dihydro-4H-cyclopenta[c]isoxazol-3-yl)-N-(-phenylmethylidene)amine of the formula 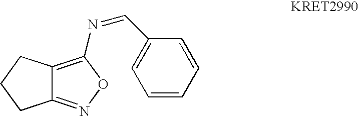
as an orange solid, m.p.=166° C.

Analogously to Examples 1 to 8 and/or in accordance with the general statements in the descriptions of the experiments, it is possible to obtain the compounds listed in Table 1.

TABLE 1

| | | Physical | Bayer |
|---|---|---|---|
| Example | Structural formula | data | Code |
| 9 | 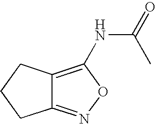 | m.p. = 151° C. | KRET2809 |
| 10 | 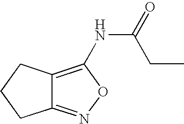 | m.p. = 109° C. | KRET2898 |
| 11 | 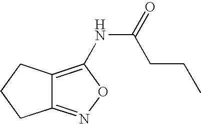 | n_{D}²⁶ = 1.4970 | KRET2908 |
| 12 | 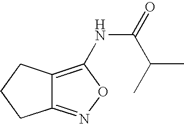 | m.p. = 116° C. | KRET2910 |
| 13 | 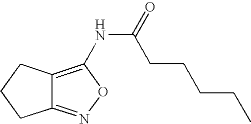 | n_{D}²³ = 1.5020 | KRET2916 |
| 14 | 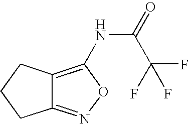 | m.p. = 118° C. | KRET2897 |
| 15 | 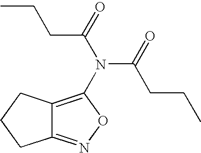 | n_{D}²⁶ = 1.4820 | KRET2817 |
| 16 | 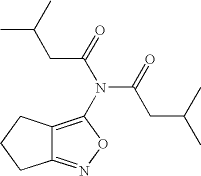 | m.p. = 35° C. | KRET2915 |
| 17 | 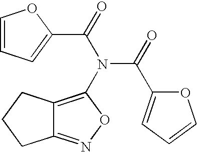 | m.p. = 150° C. | KRET3019 |
| 18 | 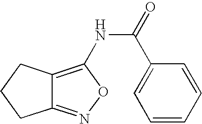 | ¹H NMR(CDCl₃) δ = 2.4(2H), 2.7(2H), 3.0(2H), 7.4-7.9(5H), 9.1(1H). | KRET2816 |
| 19 | 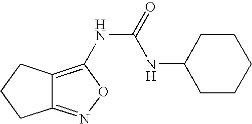 | m.p. = 152° C. | KRET2974 |
| 20 | 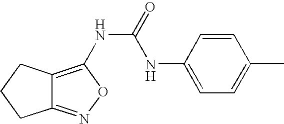 | m.p. = 218° C. | KRET2961 |
| 21 | 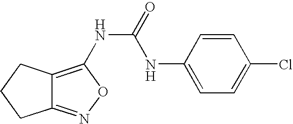 | m.p. = 225° C. | KRET2874 |
| 22 | 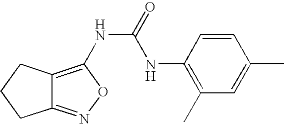 | m.p. = 230° C. | KRET2876 |
| 23 | 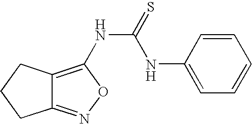 | m.p. = 146° C. | KRET2833 |
| 24 | 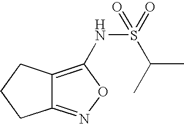 | m.p. = 91° C. | KRET2977 |
| 25 | 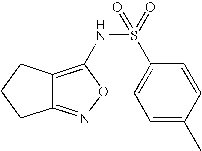 | m.p. = 112° C. | KRET2976 |
| 26 | 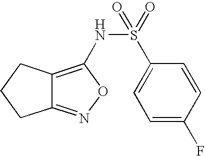 | n_{D}²⁶ = 1.5585 | KRET2993 |
| 27 | 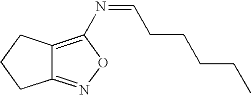 | m.p. = 125° C. | KRET3021 |
| 28 | 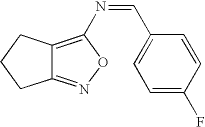 | m.p. = > 260° C. | KRET3001 |
| 29 | 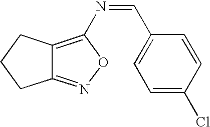 | m.p. = 190° C. | KRET3000 |
| 30 | 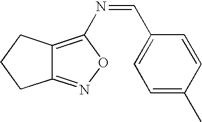 | m.p. = 238° C. | KRET2999 |
| 31 | 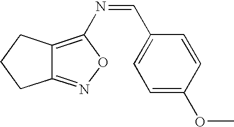 | m.p. = 249° C. | KRET3013 |
| 32 | 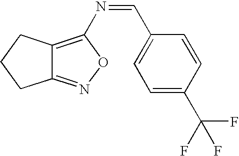 | m.p. = > 260° C. | KRET3018 |
| 33 | 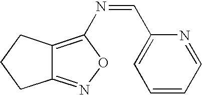 | m.p. = 167° C. | KRET3017 |

### Example 34

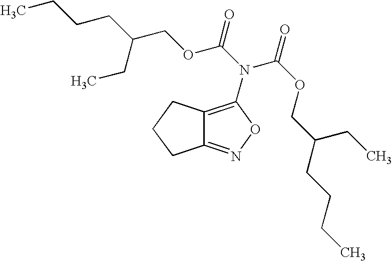

0.50 g (4.0 mmol) of 5,6-dihydro-4H-cyclopenta[c]isoxazole-3-amine and 0.78 g (6.0 mmol) of diisopropylethylamine were dissolved in 15 ml THF. The corresponding chloroformic acid (4.0 mmol) was then slowly added dropwise. Slightly exothermal reaction. A clear solution was formed. Precipitation of a white solid and stirring at RT overnight was followed by concentration. Dissolved in 11:1 hexane/ethyl acetate and column chromatography (20 Min.) on silica gel. A yellow oil was formed. Yield about 43%. Refraction: 1.4753 (29° C.); Rf value: 0.71 (1:1 hexane/ethyl acetate)

### Example 35

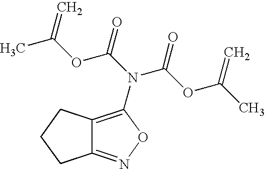

Analogous to Example 34. A colorless oil was formed. Yield about 15%. Refraction: 1.471 (29° C.); Rf value: 0.62 (1:1 hexane/ethyl acetate)

### Example 36

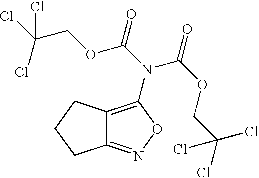

Analogous to Example 34. After concentration dissolved in 8:1 hexane/ethyl acetate and column chromatography (30 min.) using the same ratio. A colorless oil was formed which later crystallized giving a white solid. Yield about 21%. m.p.: 106° C.; Rf value: 0.65 (1:1 hexane/ethyl acetate)

### Example 37

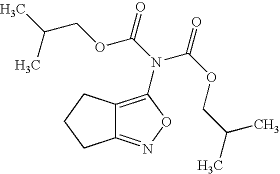

Analogous to Example 34. After 4 h, the reaction was virtually complete. Concentration. Dissolved in 8:1 hexane/ethyl acetate and column chromatography (30 min.) using the same ratio. A colorless oil was formed. Yield about 40%. Refraction: 1.465 (29° C.); Rf value: 0.67 (1:1 hexane/ethyl acetate)

### Example 38

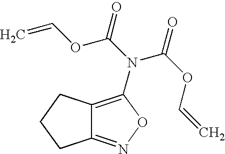

Analogous to Example 34. After 4 h, TLC showed that the reaction was virtually complete. Concentration. Dissolved in 8:1 hexane/ethyl acetate and column chromatography (30 min.) using the same ratio. A colorless oil was formed. Yield about 33%. Refraction: 1.4632 (22° C.); Rf value: 0.64 (1:1 hexane/ethyl acetate)

### Example 39

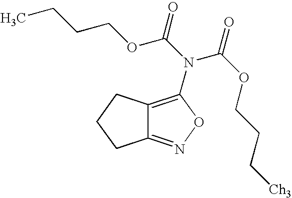

Analogous to Example 34. After 4 h, the reaction was virtually complete. Concentration. Dissolved in 8:1 hexane/ethyl acetate and column chromatography (30 Min.) using the same ratio. A colorless oil was formed. Yield about 39%. Refraction: 1.4775 (23° C.); Rf value: 0.72 (1:1 hexane/ethyl acetate)

### Example 40

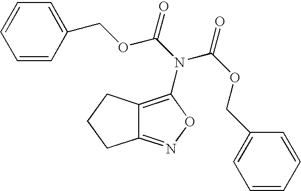

Analogous to Example 38. After concentration dissolved in 8:1 hexane/ethyl acetate and column chromatography (30 Min.) using the same ratio. A colorless oil was formed. Yield about 18%. ¹HNMR (400 MHz, CDCl₃) 7.35 (m, 8H), 7.28 (m, 2H), 5.32 (s, 2H), 5.20 (s, 2H), 3.06 (m, 2H), 3.61 (m, 2H), 2.00 (m, 2H) ppm: Rf value: 0.62 (1:1 hexane/ethyl acetate);

### Example 41

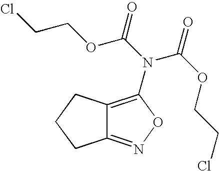

Analogous to Example 38. Dissolved in 8:1 hexane/ethyl acetate and column chromatography (30 min.) using the same ratio. A colorless oil was formed. Yield about 30%. ¹HNMR (400 MHz, CDCl₃) 4.59 (m, 2H), 4.47 (m, 2H), 3.78 (m, 2H), 3.71 (m, 2H), 3.08 (m, 2H), 2.63 (m, 2H), 2.03 (m, 2H) ppm; Rf value: 0.54 (1:1 hexane/ethyl acetate)

### Example 42

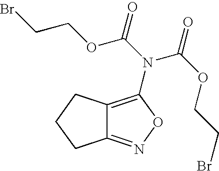

Analogous to Example 38. Dissolved in 8:1 hexane/ethyl acetate and column chromatography (30 min.) using the same ratio. An orange viscous oil was formed. Yield about 27%. Refraction: 1.4544 (29° C.); Rf value: 0.58 (1:1 hexane/ethyl acetate)

### Example 43

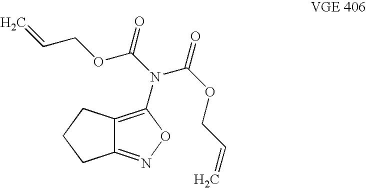

Analogous to Example 38, dissolved in 8:1 hexane/ethyl acetate and column chromatography (30 min.) using the same ratio. A virtually colorless oil was formed. Yield about 42%. Refraction: 1.4996 (23° C.); Rf value: 0.61 (1:1 hexene/ethyl acetate)

### Use Example A

To demonstrate the activity against bacteria, the minimum inhibitory concentrations (MIC) of active compounds according to the invention were determined:

A defined Landy Agar was admixed with active compounds according to the invention in concentrations of from 0.1 mg/ml to 5000 mg/ml. After the agar had solidified, it was contaminated with pure cultures of the test organisms listed in Table 2. The MIC was determined after 3 days of storage at 28° C. and 60-70% relative atmospheric humidity. The MIC is the lowest concentration of active compound at which there is no colonization by the microbial species used. The MIC values that were determined are listed in Table 2 below.

TABLE 2
Minimum inhibitory concentrations (ppm) of compounds of the
formula (I) according to the invention

| Example No. | Pseudomonas aeroginosa | Bacillus subtilis |
|---|---|---|
| 1 | <100 | <40 |
| 5 | <100 | <40 |
| 7 | 400 | 100 |
| 14 | <100 | <40 |
| 15 | <100 | 100 |
| 19 | 400 | 100 |

### Use Example B

To demonstrate the activity against fungi, the minimum inhibitory concentrations (MIC) of active compounds according to the invention were determined:

An agar which had been prepared using malt extract was admixed with active compounds according to the invention in concentrations of from 0.1 mg/l to 5000 mg/l. After the agar had solidified, it was contaminated with pure cultures of the test organisms listed in Table 3. The MIC was determined after 3 weeks of storage at 28° C. and 60-70% relative atmospheric humidity. The MIC is the lowest concentration of active compound at which there is no colonization by the microbial species used. The MIC values that were determined are listed in Table 3 below.

TABLE 3
Minimum inhibitory concentrations (ppm) of compounds of the
formula (I) according to the invention

| | Penicillium | Chaetomium | Aspergillus |
|---|---|---|---|
| Example No | brevicaule | globosum | niger |
| 1 | <100 | <100 | <100 |
| 5 | 100 | 100 | 200 |
| 14 | 100 | 100 | 100 |
| 15 | 200 | 100 | >400 |

### Use Example C

### Example of Antifungal Action

Analogously to Use Example B, the minimum inhibitory concentrations (MIC) of active compounds according to the invention were determined. Against fungi such as Fusarium solani, Geotrichum candidum and Rhodotorula rubra the compounds tested had minimum inhibitory concentrations of <500 ppm:

TABLE 4

| Example | Rhodotorula | Fusarium | Geotrichum |
|---|---|---|---|
| No. | rubra | solani | candidum |
| 42 | 500 ppm | 200 ppm | 500 ppm |
| 41 | 200 ppm | 200 ppm | 500 ppm |
| 38 | 100 ppm | 50 ppm | 100 ppm |

### Use Example D

### Example of Antibacterial Action

Analogously to Use Example A, the minimum inhibitory concentrations (MIC) of active compounds according to the invention were determined. Both on chemically defined medium and on complex medium, the compounds tested had minimum inhibitory concentrations </=100 ppm against Pseudomonas aeruginosa NCIB 6749:

TABLE 5

| Example | MIC values | MIC values |
|---|---|---|
| No. | (complex medium) (ppm) | (chem.def. medium) (ppm) |
| 43 | 10 | 20 |
| 42 | 5 | 10 |
| 41 | 5 | 10 |
| 40 | 20 | 20 |
| 36 | 5 | 10 |
| 35 | 10 | 10 |
| 39 | 10 | 10 |
| 38 | 20 | 50 |
| 37 | 5 | 10 |
| 34 | 50 | 100 |

1. A compound of the formula (I) 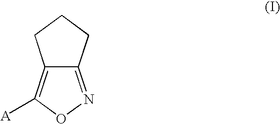
in which
A represents a radical radical 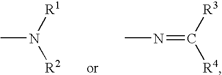
in which
R¹ and R² independently of one another represent hydrogen, halogen, cyano, nitro or represent in each case optionally substituted alkyl, alkenyl, alkynyl, aryl, heterocyclyl, —COR⁵, —CONR⁶, —CSNR⁷ or —SO₂R⁸,
where
R⁵ to R⁸ independently of one another represent in each case optionally substituted alkyl, alkenyl, alkynyl, aryl or heterocyclyl,
and
R³ and R⁴ independently of one another represent hydrogen, or represent in each case optionally substituted alkyl, alkenyl, alkynyl, aryl and heterocyclyl,
or a salt or acid addition compound thereof.

2. A compound as claimed in claim 1, characterized in that
R¹ and R² independently of one another represent hydrogen, halogen, cyano, nitro or in each case optionally substituted C₁-C₈-alkyl, C₂-C₈-alkenyl, C₂-C₈-alkynyl, phenyl or heterocyclyl, or represent a radical —COR⁵, CONR⁶, —CSNR⁷ or —SO₂R⁸,
where
R⁵ to R⁸ independently of one another represent hydrogen, halogen, cyano, nitro or represent in each case optionally substituted C₁-C₈-alkyl, C₂-C₈-alkenyl, C₂-C₈-alkynyl, phenyl or heterocyclyl, and
R³ and R⁴ independently of one another represent hydrogen, halogen, cyano, nitro or represent in each case optionally substituted C₁-C₈-alkyl, C₂-C₈-alkenyl, C₂-C₈-alkynyl, phenyl or heterocyclyl.

3. A process for preparing compounds of the the formula (I) as claimed in claim 1
in which
A represents a radical 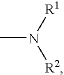
and
R¹ and R² represent hydrogen,
characterized in that hydroxylamine or its salts are reacted with 2-amino-1-cyclopentene-1-carbonitrile, if appropriate in the presence of diluents and if appropriate in the presence of a catalytic or stoichiometric amount of base.

4. A process for preparing compounds of the formula (I) as claimed in claim 1
in which
A represents a radical 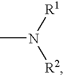
and
R¹ and R² independently of one another represent halogen, cyano, nitro or represent in each case optionally substituted alkyl, alkenyl, alkynyl, aryl, heterocycyl, —COR⁵, —CONR⁶, —CSNR⁷ or —SO₂R⁸,
and
R⁵ to R⁸ are as defined in claim 1,
characterized in that compound of the formula (I)
in which
A represents a radical 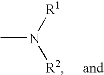
R¹ and R² represent hydrogen, is reacted
a) with carboxylic anhydrides of the formula (II), 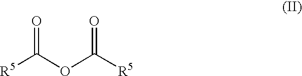
in which
R⁵ is as defined in claim 1
or
b) with carbonyl halides of the formula (III) 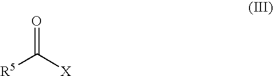
in which
R⁵ is as defined in claim 1 and X represents Cl and Br,
or
c) with isocyanates of the formula (IV) 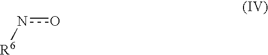
in which
R⁶ is as defined in claim 1
or
d) with isothiocyanates of the formula (V) 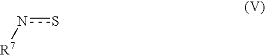
in which
R⁷ is as defined in claim 1
or
e) with sulfonyl chlorides of the formula (VI) 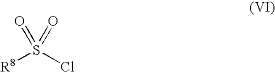
in which
R⁸ is as defined in claim 1,
if appropriate in the presence of diluents and if appropriate in the presence of a catalytic or stoichiometric amount of base.

5. A process for preparing compounds of the formula (I) as claimed in claim 1
in which
A represents 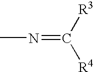
and
R³ and R⁴ are as defined in claim 1,
characterized in that compound of the formula (I)
in which
A represents 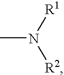
and R¹ and R² represent hydrogen,
is reacted with aldehydes or ketones of the formula (VII) 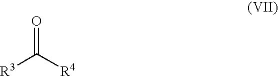
in which
R³ and R⁴ are as defined in claim 1,
if appropriate in the presence of diluents and if appropriate in the presence of a catalytic or stoichiometric amount of base.

6. A microbicidal composition, comprising at least one compound as claimed in at least one of claims 1 and 2 and at least one solvent or diluent and also, if appropriate, processing auxiliaries and, if appropriate, further antimicrobially active compounds.

7. A composition as claimed in claim 6, characterized in that it comprises at least one further antimicrobially active compound from the group of the fungicides, bactericides, herbicides and/or insecticides.

8. The use of compounds as claimed in at least one of claims 1 and 2 as a microbicide for protecting industrial materials.

9. The use as claimed in claim 8, characterized in that the industrial materials are adhesives, sizes, paper, board, leather, wood, timber products, paints, cooling lubricants and heat-transfer liquids.

10. A method for protecting industrial materials against infestation and/or destruction by microorganisms, characterized in that at least one compound as claimed in at least one of claims 1 and 2 is allowed to act on the microorganism or its habitat.

11. An industrial material, comprising at least one compound as claimed in at least one of claims 1 and 2.
